(19) United States
(12) Patent Application Publication
(10) Pub. No.: US 2013/0198991 A1
(43) Pub. Date: Aug. 8, 2013

(54) ATTACHABLE UV LIGHT FOR CLEANING TOOLS

(71) Applicant: Lloyd Cadigan, Millstone Township, NJ (US)

(72) Inventor: Lloyd Cadigan, Millstone Township, NJ (US)

(21) Appl. No.: 13/366,345

(22) Filed: Feb. 5, 2012

(51) Int. Cl. A47L 9/00; A47L 13/50

(52) U.S. Cl. 15/246.3; 15/246

(57) ABSTRACT

A household cleaning tool enabled with UV light unit. A conventional cleaning tool head is attached with a UV light unit through an industrial strength sticky tape or other means of attachment. The UV light unit has a safety cover. The UV light unit is detachable after using.

### DESCRIPTION OF RELATED ART

The present application relates to a ultraviolet light, and more particularly to a UV light set attachable to a cleaning tool.

Note that the points discussed below may reflect the hindsight gained from the disclosed inventions, and are not necessarily admitted to be prior art.

Handheld UV lights have been developed for sanitizing purposes, especially been used in hospitals. However, UV lights have not been widely used in household cleaning. Carpets, mattresses, long timed stored clothing are prone for microorganism growth. Traditionally housewives would take out these items to bask under the UV lights of the Sun in the summer for sanitizing. Efforts have been made to incorporate UV lights into household vacuums. For example, US 2011/0214686 A1 patent application publication describes a vacuum cleaner that has a built-in UV light source at the rear chamber of the vacuum. Another European patent application publication EP 2243414 A2 has a built-in UV-light source at the front section of the vacuum chamber. WO 2006/121286 A1 discloses a complicated vacuum design that incorporates a UV light source.

However, UV light is not safe to use in an ordinary household. UV lights can hurt eyes and skins if being exposed directly. The cleaning tools that directly incorporate UV light in its structure are not easy to safety control for household use. A detachable UV light is in much better position for safety reasons. Adults can decide a specific time to use UV light for sanitizing and detach the UV light for safe keeping. The UV light detached vacuum can be used for the usual cleaning without much safety concern. A detachable UV light design provides an economical solution for household sanitizing since no new vacuum needs to be purchased.

### SUMMARY

The present application discloses an improved UV light design that is attachable to a vacuum or other cleaning tools, such as a broom.

In one embodiment, a UV light is fixedly amounted in a holding box with an extensible cover that can be pulled out to cover the UV light bulb for safety and storage.

In one aspect of an embodiment, an extensible cover comprises two panels foldably mounted together, the holding box is opened by folding the two panels together and by sliding the folded panels into the top side of the holding box.

In one aspect of an embodiment, a commercially available UV light bulb is attached into the holding box with a pair of Velcro® industrial strength sticky back tape strands or other means of attachment.

In one aspect of an embodiment, a holding box is attached to the front edge of a vacuum through a pair of Velcro® industrial strength sticky back tape strands or other means of attachment.

In one aspect of an embodiment, a commercially available UV light is directly attached to the front edge of a vacuum through a pair of Velcro® industrial strength sticky back tape strands or other means of attachment.

In one aspect of an embodiment, a commercially available UV light is directly attached to the front edge of an elongated pole with a cross bar sweeper at the lower end of the pole through a pair of Velcro® industrial strength sticky back tape strands or other means of attachment.

The disclosed innovation, in various embodiments, provide an easy and economical alternative for household cleaning and sanitization needs.

### BRIEF DESCRIPTION OF THE DRAWINGS

The disclosed application will be described with reference to the accompanying drawings, which show important sample embodiments of the invention and which are incorporated in the specification hereof by reference, wherein:

FIG. 1A shows a prospective view of an example holding box with an extended cover in accordance with this application.

FIG. 1B shows a prospective view of an example holding box with the cover folded in accordance with this application.

FIG. 1C shows a prospective view of the rear side of an example holding box in accordance with this application.

FIG. 2 shows a prospective view of a UV light bulb in accordance with this application.

FIG. 3 shows a prospective view of a UV light unit together with a conventional vacuum cleaning head in accordance with this application.

FIG. 4 shows a prospective view of a conventional vacuum cleaning head attached with a UV light unit in accordance with this application.

FIG. 5 shows a prospective view of a elongated pole with a cross bar sweeper at the lower section of the pole where by a UV light unit can be attached in accordance with this application.

### DETAILED DESCRIPTION OF SAMPLE EMBODIMENTS

The numerous innovative teachings of the present application will be described with particular reference to presently preferred embodiments (by way of example, and not of limitation). The present application describes several embodiments, and none of the statements below should be taken as limiting the claims generally.

For simplicity and clarity of illustration, the drawing figures illustrate the general manner of construction, and description and details of well-known features and techniques may be omitted to avoid unnecessarily obscuring the invention. Additionally, elements in the drawing figures are not necessarily drawn to scale, some areas or elements may be expanded to help improve understanding of embodiments of the invention.

The terms “first,” “second,” “third,” “fourth,” and the like in the description and the claims, if any, may be used for distinguishing between similar elements and not necessarily for describing a particular sequential or chronological order. It is to be understood that the terms so used are interchangeable. Furthermore, the terms “comprise,” “include,” “have,” and any variations thereof, are intended to cover non-exclusive inclusions, such that a process, method, article, apparatus, or composition that comprises a list of elements is not necessarily limited to those elements, but may include other elements not expressly listed or inherent to such process, method, article, apparatus, or composition.

It is contemplated and intended that the disclosed application also be applied to broom and other suitable cleaning tools; for clarity reason, the examples are given based on converting conventional vacuum cleaner into a UV light enabled vacuum cleaner.

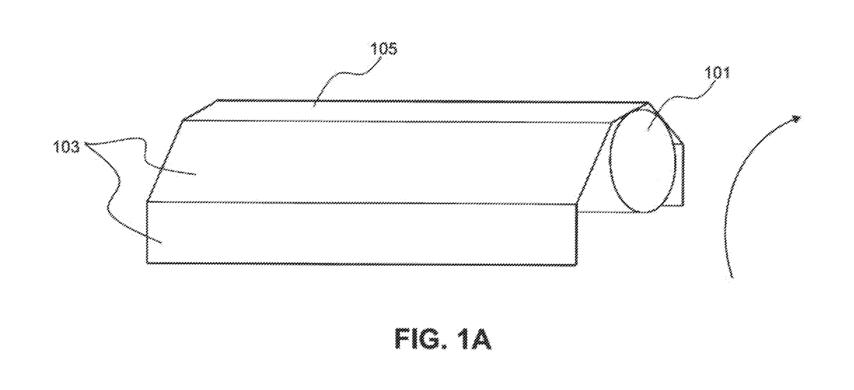

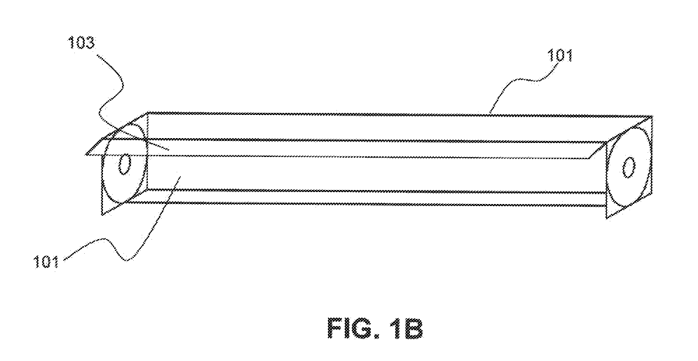

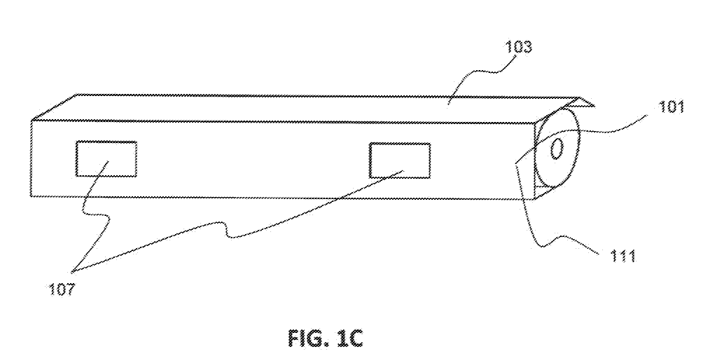

In reference to FIGS. 1A, 1B, and 1C, a vacuum attachable UV light unit comprises an elongated cuboidal holding box 105 and a UV light bulb set 101 attachedly mounted inside holding box 105. To protect from being burned by UV light when turning on the UV light, holding box 105 comprises an foldable cover 103. Preferably cover 103 comprises two panels mounted together so that they can be folded together. Cover 103 is slidely mounted to the top side wall 109 of holding box 105 so that when using the UV light for cleaning, cover 103 can be lifted up and folded underneath the top side wall. Alternatively, cover 103 may be folded onto the top of the top side wall.

To attach the UV light unit to a cleaning tool, two sets of Velcro® industrial strength sticky back tape strands 107 are attached to back wall 111 of holding box 105. Folding box 105 also includes the necessary power connections for the UV light bulb set.

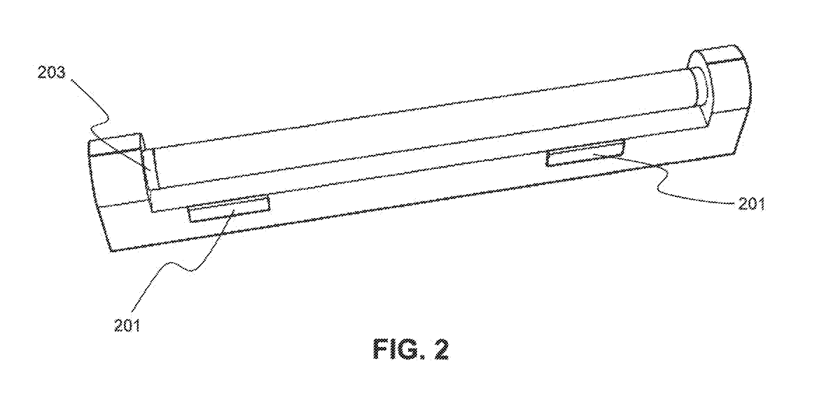

In reference to FIG. 2, alternatively two sets of Velcro® industrial strength sticky back tape strands 201 may be attached directly to a UV light bulb set 203 which can be directly attached to the vacuum front side.

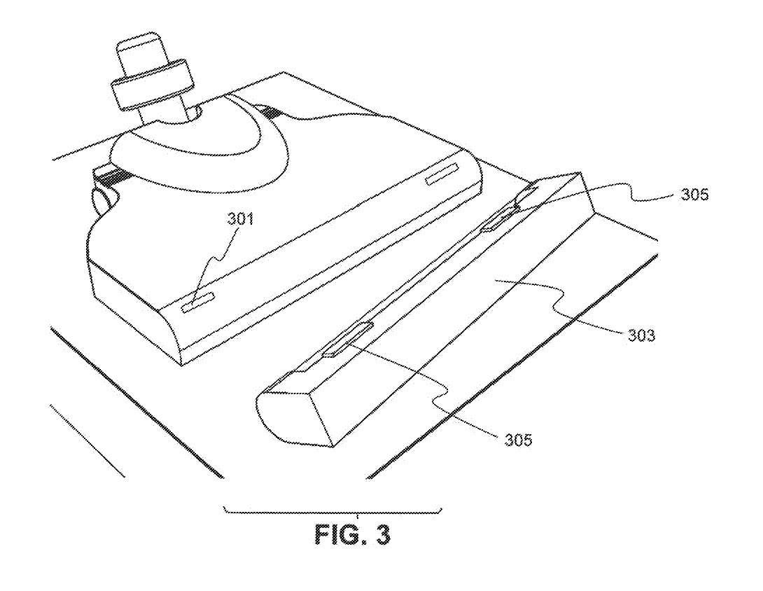

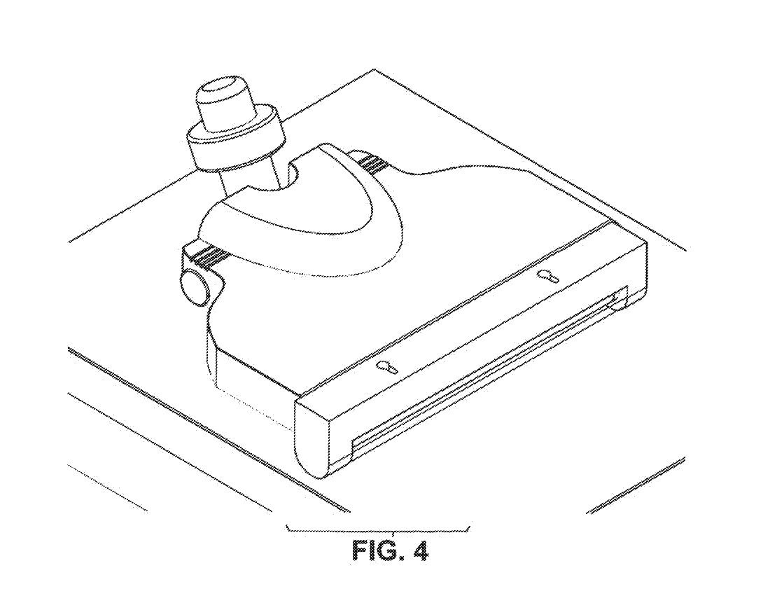

In reference to FIGS. 3 and 4, two sets of Velcro® industrial strength sticky back tape strands 305 are attached to UV light unit 303, and two sets are attached to vacuum head front edge 301, a UV light enabled vacuum is assembled as shown in FIG. 4 by sticking the vacuum and UV light unit 303 together. The UV light may be powered by battery or separate electricity power plug.

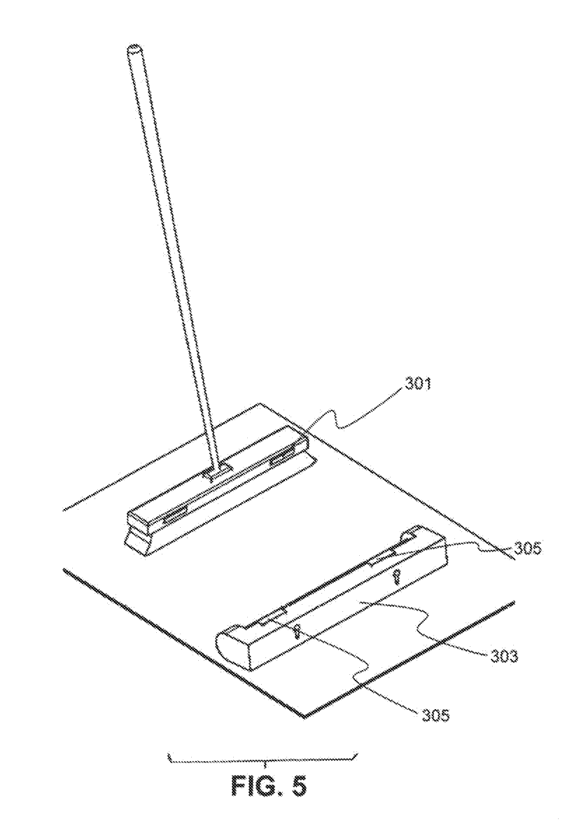

In reference to FIG. 5, two sets of Velcro® industrial strength sticky back tape strands 305 are attached to UV light unit 303, and two sets are attached to elongated pole with cross bar sweeper at front edge 301, a UV light enabled sweeper is assembled as shown in FIG. 5 by sticking the broom and UV light unit 303 together. The UV light may be powered by battery or separate electricity power plug.

As will be recognized by those skilled in the art, the innovative concepts described in the present application can be modified and varied over a tremendous range of applications, and accordingly the scope of patented subject matter is not limited by any of the specific exemplary teachings given. It is intended to embrace all such alternatives, modifications and variations that fall within the spirit and broad scope of the appended claims.

Additional general background, which helps to show variations and implementations, may be found in the following patent application publications, all of which are hereby incorporated by reference herein for all purposes: US 2011/0214686 A1, EP 2243414 A2 and WO 2006/121286 A1.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC section 112 unless the exact words “means for” are followed by a participle.

The claims as filed are intended to be as comprehensive as possible, and NO subject matter is intentionally relinquished, dedicated, or abandoned.

What is claimed is:

1. A household cleaning tool head enabled with a detachable UV light unit, comprising:
a household cleaning tool head attached with a double side industrial strength sticky tape or other means of attachment; and
a UV light unit attached with a double side industrial strength sticky tape or other means of attachment,
wherein said household cleaning tool head and said UV light unit are stuck together by said sticky tape or other means of attachment in a way so that said UV light is operated to sanitize when using said cleaning tool for household cleaning.

2. The household cleaning tool head enabled with a detachable UV light unit of claim 1 wherein said household cleaning tool is a vacuum head.

3. The household cleaning tool head enabled with a detachable UV light unit of claim 1 wherein said sticky tape is a Velcro® industrial strength sticky back tape or other means of attachment.

4. The household cleaning tool head enabled with a detachable UV light unit of claim 1 wherein said UV light unit includes a holding box that has a foldable cover for safety.

5. The household cleaning tool head enabled with a detachable UV light unit of claim 4 wherein said holding box is plastic.
